(19) United States
(12) Patent Application Publication
(10) Pub. No.: US 2022/0296750 A1
(43) Pub. Date: Sep. 22, 2022

(54) CHEMICAL-FREE DRY HEAT AND UVC DECONTAMINATION UNIT AND METHOD

(71) Applicant: Crescent Metal Products, Inc., Mentor, OH (US)

(72) Inventors: Adolph DeGennaro, Fairport Harbor, OH (US); Yogesh Ramadoss, Cleveland Heights, OH (US); Gregory Baggott, Chardon, OH (US)

(21) Appl. No.: 17/696,305

(22) Filed: Mar. 16, 2022

Related U.S. Application Data
(60) Provisional application No. 63/162,105, filed on Mar. 17, 2021.

(51) Int. Cl. A61L 2/10; A61L 2/28; A61L 2/04

(52) U.S. Cl. CPC A61L 2/10; A61L 2/28; A61L 2/04; A61L 2202/14; A61L 2202/11; A61L 2202/122; A61L 2202/16

(57) ABSTRACT

A decontamination unit includes a housing having an internal cavity for containing an object that is to be decontaminated, a heater that is configured to heat the internal cavity and object, and at least one light source configured to supply ultraviolet light, such as UVC, to the internal cavity to irradiate the object. A decontamination cycle may perform the heat cycle and/or the UV cycle, which may be performed simultaneously during at least a part of the cycle to enhance the effectiveness in decontaminating the object.

### RELATED APPLICATIONS

This application claims the benefit of U.S. Provisional Application No. 63/162,105 filed Mar. 17, 2021, which is hereby incorporated herein by reference in its entirety.

### TECHNICAL FIELD

The present invention relates generally to decontamination units, and more particularly, to a chemical-free dry heat and ultraviolet-C (UVC) decontamination unit and method.

### BACKGROUND

Decontamination units generally involve a process of eliminating contaminants from an object, such as the destroying or removal of micro-organisms, viruses, bacteria, or the like, some of which may be harmful to human health. There are various types of decontamination units depending on the contaminant to be removed.

### SUMMARY

Using ultraviolet-C light (UVC) is one conventional approach for decontaminating or disinfecting objects. The wavelength of UVC generally is in the range of 200 nm to 280 nm, which when absorbed by a living organism such as bacteria or viruses will destroy the organism's cells, rendering it unable to reproduce. However, the effectiveness of UVC is based on the ability of the UVC to penetrate the cells, and thus generally involves direct line of site to the ultraviolet rays. As such, regions of the contaminated object facing away from the light source or covered by a shadow or other object may not be disinfected appropriately.

According to an aspect, a decontamination unit and method is provided herein that enhances the effectiveness of UV-based decontamination by combining the UV decontamination cycle with dry heat.

According to another aspect, a decontamination unit and method is provided herein that enables the use of both heat and UV light to decontaminate an object, either separately or in combination.

More particularly, according to an aspect, a unique dry heat and/or UV-light decontamination unit and method is provided herein that includes one or more advantages of: (i) a relatively low-temperature dry heat decontamination cycle combined with an associated level of UV-light, such as UVC, for decontaminating an object; (ii) decontamination at atmospheric pressure; (iii) decontamination without the use of chemicals, which may be particularly advantageous for electronic devices, for example; (iv) an arrangement of the UV light sources relative to the object-supports that minimizes shadowing and enhances decontamination; (v) a relatively short cycle time for increasing decontamination throughput of contaminated objects, which may be particularly advantageous in the event of a pandemic, for example; and/or (vi) a relatively inexpensive unit that is affordable and deployable at various points of use, such as at schools, police stations, fire stations, department of defense, other government agencies, elderly care facilities, hospitals, or the like.

According to an aspect, a decontamination unit includes: a housing having an internal cavity and an opening for accessing the internal cavity; a door coupled to the housing for opening or closing the opening for permitting or restricting access to the internal cavity; a heater in thermal communication with the internal cavity and configured to heat the internal cavity; and a light source configured to supply ultraviolet light to the internal cavity; wherein the decontamination unit is configured to enable each of (i) heating the internal cavity with the heat source, and (ii) irradiating the internal cavity with the light source.

According to another aspect, a method of decontaminating an object, includes: placing the object within an internal cavity of a decontamination unit; supplying heat to the internal cavity and increasing the temperature of the internal cavity to a temperature value in a range from 100° F. to 220° F.; supplying UV light to the internal cavity; and performing decontamination with the supplying heat and the supplying UV light for a period of time in a range from 15 minutes to 60 minutes.

The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention.

FIG. 1 is a front elevational view of an exemplary dry heat and ultraviolet (UV) light decontamination unit according to an embodiment.

FIG. 2 is a rear elevational view of the decontamination unit.

FIG. 3 is a side elevational view of the decontamination unit.

FIG. 4 is a front elevational view of the decontamination unit with the door removed and showing an internal cavity of the unit.

FIG. 5 is a perspective view of the decontamination unit with a top cover of the housing removed, and a rear panel containing UV light sources shown in exploded view.

FIG. 6 is an exemplary schematic view of the decontamination unit shown in FIG. 1.

FIG. 7 is a flow chart showing an exemplary decontamination process according to an embodiment.

FIG. 8 is a front elevational view of another exemplary dry heat and UV light decontamination unit according to another embodiment.

FIG. 9 is a side elevational view of the decontamination unit in FIG. 8.

FIG. 10 is a rear elevational view of the decontamination unit in FIG. 8.

FIG. 11 is a front elevational view of the decontamination unit in FIG. 8 with the door removed and showing an internal cavity of the unit.

### DETAILED DESCRIPTION

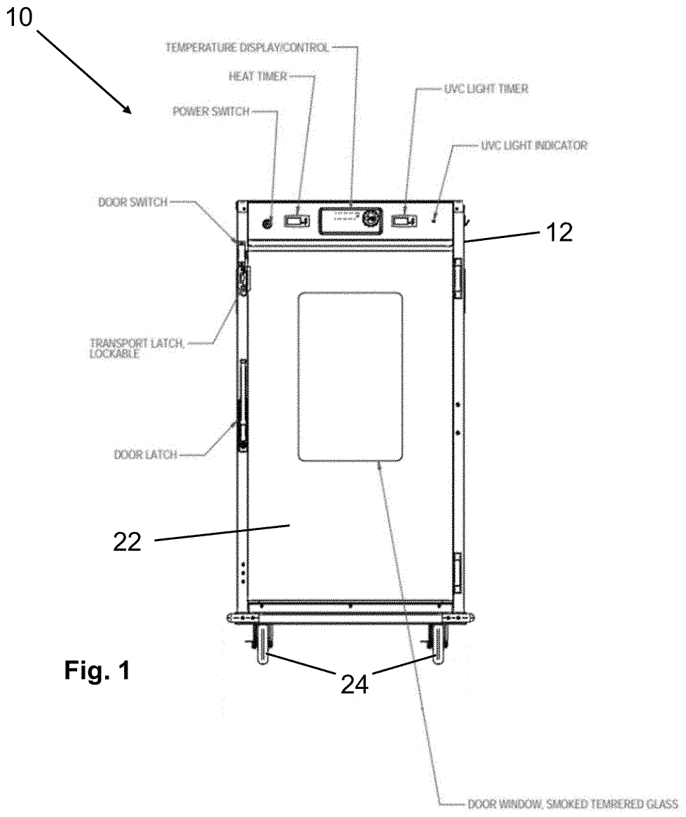

Referring to the drawings, and initially to FIGS. 1-6, an exemplary decontamination unit 10 is shown. The decontamination unit 10 includes a housing 12 having an internal cavity 14 for containing a contaminated object 15 that is to be decontaminated. The decontamination unit 10 also includes a heater 16 that is in thermal communication with the internal cavity 14 and is configured to heat the internal cavity 14 and object 15, preferably with dry heat. The decontamination unit 10 also includes at least one light source 18 configured to supply ultraviolet light (e.g., UVC light) to the internal cavity to irradiate the object 15. A controller 20 is provided that is configured to control a temperature and preferably also the UV irradiation/exposure within the internal cavity 14 according to a decontamination cycle for decontaminating the object 15. The decontamination cycle may be performed at atmospheric pressure. In exemplary embodiments, the decontamination cycle may be performed free from the use of chemicals.

In exemplary embodiments, the housing 12 includes a base and a main body, which may be composed of one or more parts. The housing 12 may be reinforced by a frame, such as an internal stainless-steel frame, and may be made of any suitable material or combination of materials. One or more doors 22 are provided for opening or closing an opening 23 or openings in the front of the housing 12 for allowing access to the internal cavity 14.

The housing 12 and the door 22 may be insulated to maintain the desired temperature range inside of the cavity 14 and/or prevent temperature loss. The housing 12 may include an outer housing portion, which forms an outer surface of the housing, and an inner housing portion, which forms an inner surface of the internal cavity 14 (also referred to as internal chamber 14). The outer housing portion may be made of any suitable material, such as powder-coated aluminum. The inner housing portion may be made of any suitable material, such as stainless steel, and may include smooth interior coved corners to prevent buildup of material. An insulation gap may be formed between the outer housing portion and the inner housing portion, in which the insulation gap may be filled with an insulation material, such as fiberglass and/or foam. The door 22 may be made of stainless steel and may include a gasket such as a magnetic santoprene.

The door 22 is coupled to the housing 12 by any suitable means, such as by hinges. The door 22 includes a handle configured to latch to the housing 12 by a suitable latch, such as a magnetic latch. The door can be equipped with a any suitable lock to lock the decontamination unit 10, for example during transportation or during a decontamination cycle. The door 22 also may include a viewing window, which may be made of any suitable material, such as glass, acrylic glass, etc., and preferably a UV-proof glass.

In exemplary embodiments, the decontamination unit 10 (also referred to as a cabinet, or simply unit 10) may be configured to be portable. In the illustrated embodiment, for example, a plurality of wheels 24 are provided at a bottom side of the base that allow the decontamination unit 10 to be moved easily, even when fully loaded. A brake may be provided on one or more of the wheels 24 so that the cabinet can be locked in place when being used. In the illustrated embodiment, the front two wheels are swivel casters with brakes, and the rear two wheels are fixed to provide mobility when fully loaded.

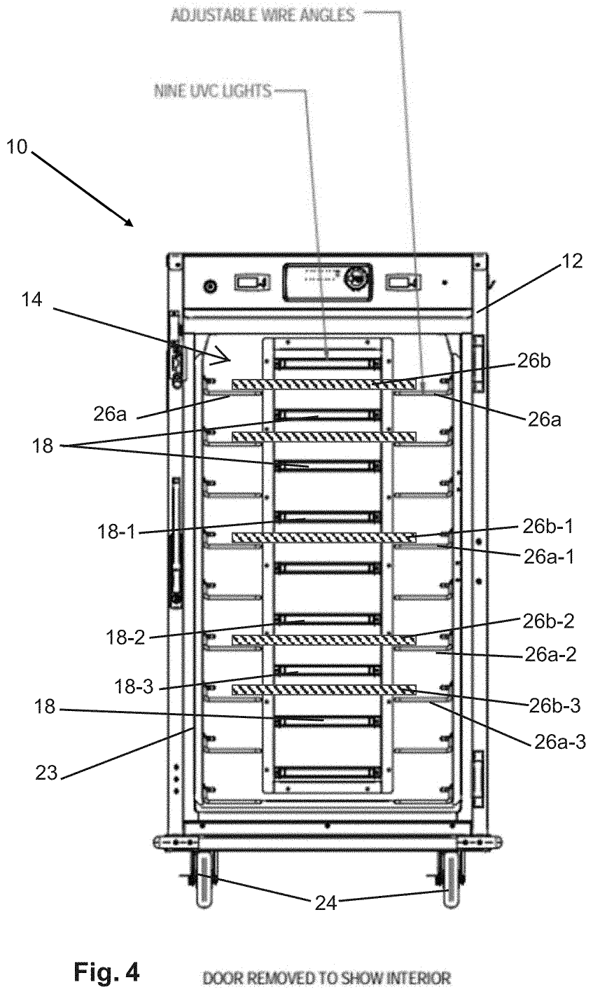

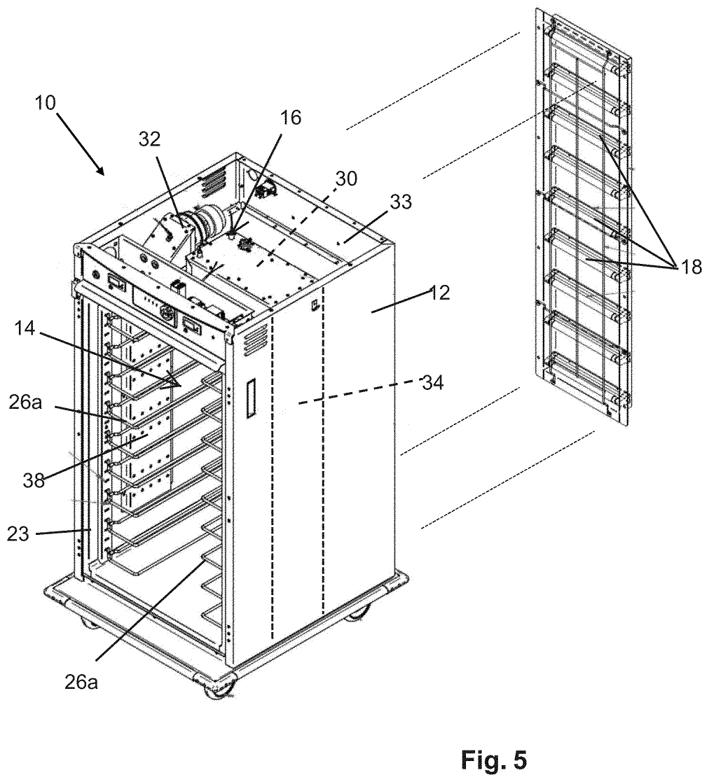

Referring particularly to FIGS. 4 and 5, the decontamination unit 10 is shown with the front door 22 removed. As shown, the unit 10 may include any suitable type or amount of object supports 26 in any suitable configuration for holding the objects to be decontaminated. For example, in the illustrated embodiment, the decontamination unit 10 may include laterally spaced apart pairs of supports 26a, such as rails, slots, holes or angles, within the internal cavity 14 for supporting laterally extending supports 26b-1, 26b-2 (depicted schematically in broken line and collectively referred to with reference numeral 26b). The laterally extending supports 26b may have any suitable structure, which may be in the form of shelves, racks and/or trays, for example. The supports 26 may be arranged vertically spaced apart from each other in the internal cavity 14. In exemplary embodiments, the various supports 26 can be adjusted and configured in any suitable manner to handle smaller objects and/or larger items.

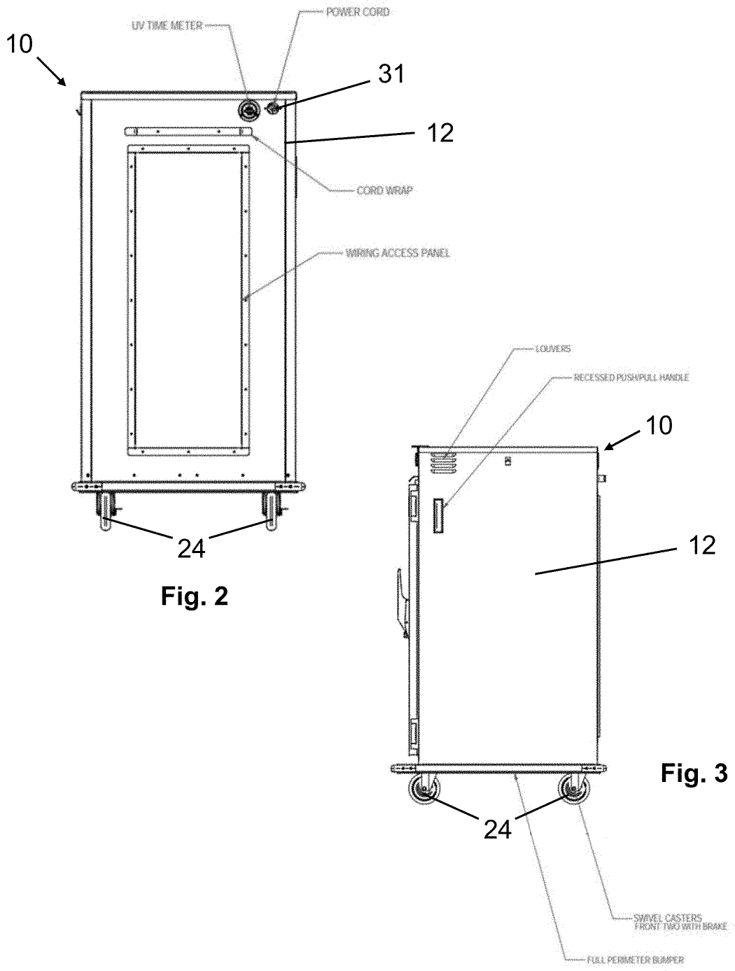

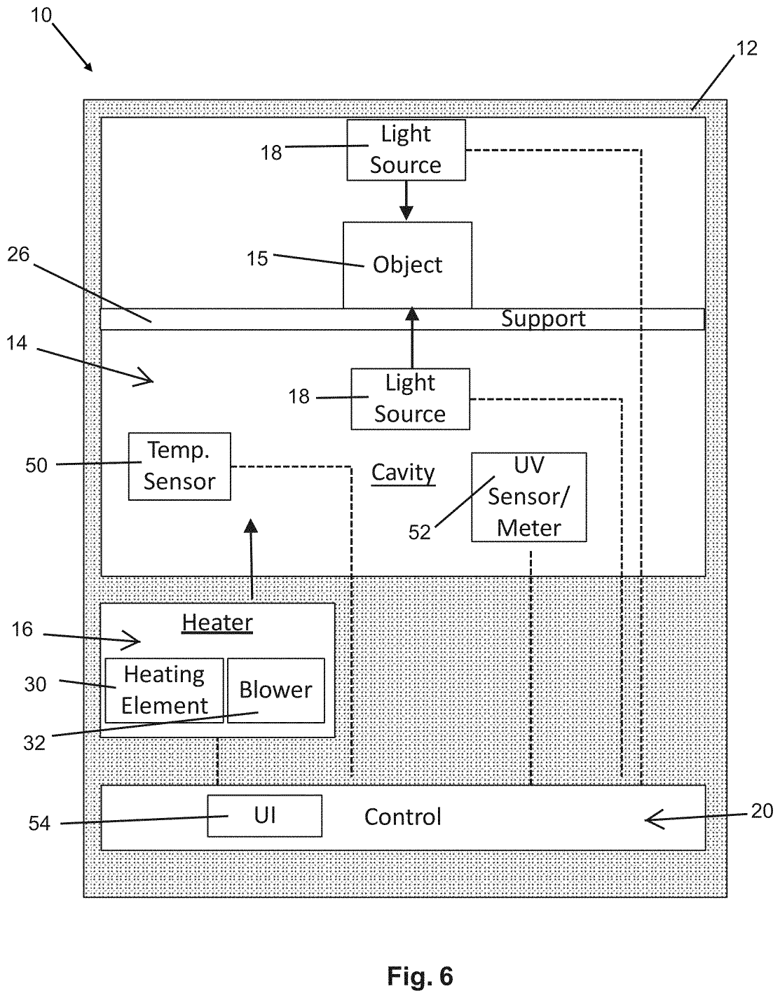

Referring to FIGS. 5 and 6, with further reference to FIG. 4, the heater 16 of the decontamination unit 10 is shown. The heater 16 may be any suitable heater capable of heating the internal cavity 14 as desired, which may include one or more of convection, conduction, and radiation heating. In the illustrated embodiment, the heater 16 includes an electric heating element 30 that is electrically coupled to a power source by an electrical cord 31 (FIG. 2). The electric heating element 30 may be any suitable heating element, such as a resistive heating element, for example. In exemplary embodiments, the heating element 30 is disposed in a cavity outside of the internal cavity 14 and heats the internal cavity 14 via convective heating. In the illustrated embodiment, for example, the heating element 30 is disposed in an upper cavity 33 (FIG. 5) above the internal cavity 14.

To provide convective heating to the internal cavity 14 via the heating element 30, a blower 32 is configured to blow air across the heating element 30 and into the internal cavity 14. The blower 32 may be any suitable type of blower at any suitable location in the decontamination unit 10 for transferring heated air from around the heating element 30 into the internal cavity 14. In the illustrated embodiment, for example, the blower 32 is disposed in the upper cavity 33 (FIG. 5) where air from the blower blows across the heating element 30 and travels through a vertical supply channel, or air passage (shown at 34) where the hot air is forced into the internal cavity 14 via openings (inlets) in the vertical supply channel 34. On another side (e.g., opposite side) of the internal cavity 14, a vertical return channel, or air passage (shown at 38) is provided having a plurality of openings (outlets) for drawing the air from the internal cavity 14 into the return channel 38. The blower 32 is operatively connected to the return channel 38 and sucks the return air back through the blower 32 and across the heating element 30. In this manner, the air within the decontamination unit 10 is recycled and is not exhausted until the door 22 is opened. Suitable seals may be provided to substantially maintain the hot air within the unit 10.

Still referring particularly to FIGS. 4-6, the at least one light source 18 of the decontamination unit 10 also is shown. In exemplary embodiments, the decontamination unit 10 includes a plurality of light sources 18, as shown. The light source or light sources 18 may be any suitable device(s) capable of supplying the desired wavelength or wavelengths of light to the internal cavity 14 for irradiating the contaminated object 15, as may be desired for particular application(s). In exemplary embodiments, the light source(s) 18 emit UV-light, and more particularly are UVC light source(s) configured to provide ultraviolet-C light in a wavelength range from 200 nm to 300 nm; more particularly, in a range from 200 nm to 280 nm; and even more particularly at about 254 nm, including all values and ranges intervening the stated values. Generally, the UV light (e.g., UVC) works by exposing or “dosing” a biological organism (e.g., virus, bacteria, etc.) with the UV light where it is absorbed by DNA, RNA, and proteins. This absorption can lead to rupture of cell walls and death of the organism, or may inactivate the DNA or RNA strands to prevent replication of the cell. Although reference is made herein predominantly to UV or UVC, it is understood that other wavelengths of light also may be used depending on the desired decontamination, including other forms of UV light (UVA, UVB, etc.) for example. As such, any reference to UVC made herein can be substituted with, or used in conjunction with, other forms of light.

Each UV light source 18 generally may include a filament, gas, coating, layer (e.g., phosphor coating of a bulb, or phosphor layer of an LED) and/or any other suitable device or material for generating the UV light. The UV light source 18 may be embodied in a suitable lamp or bulb (also 18), which may have any suitable form (e.g., mercury lamp, fluorescent bulb, LED, fiber optic cable, etc.), and which is electrically connected to a power source, such as an external power source. In exemplary embodiments, the light source, light bulb, lamp, optical cable, or other suitable device or output end of such device (hereinafter collectively referred to simply as the light source 18) may be at least partially located within the internal cavity 14 for providing direct line of sight with the object 15 or objects to be decontaminated. As shown, multiple such light sources 18 may be provided to improve exposure and coverage of the UV light, with each light source 18 being configured to irradiate a respective portion of the cavity 14 and object 15. A guard, such as a cover or wire enclosure, may enclose the output end of the light source 18 to prevent damage.

Because a plurality of supports 26 may be provided to support a plurality of objects 15 for decontamination, each support 26 in the cavity may have associated with it at least one light source 18. This better ensures that the UV light will reach the object 15 to be decontaminated, without shadowing, diffusion, or other factors that might reduce decontamination efficiency of the UV light. As shown in the illustrated embodiment, for example, a plurality of horizontal racks 26b-1, 26b-2, etc., may rest on corresponding side supports 26a (e.g., angles, slots or channels), such that the horizontal supports 26b are vertically spaced apart from each other in the internal cavity 14. At least one light source (18-1, 18-2, 18-3, etc.) is associated with each corresponding support (26b-1, 26-2, 26-3, etc.), for example at an elevation above each support (26b-1, 26-2, 26-3, etc.) to direct light on the object without shadowing or diffusion. The light source 18 below each support 26b also may direct UV light to an underside of the object(s) 15 on each support 26b. To better enable exposure of such light to the underside of the object(s) 15, the support(s) 26b may have openings and/or may be transparent to the wavelength of the UV light.

Although the illustrated embodiment only shows light sources 18 vertically spaced apart on the back wall of the cavity 14, it is understood that light sources 18 may be provided on any surface within the cavity 14 in any pattern or configuration. For example, light sources 18 may be provided on the back wall, left and right side walls, ceiling, floor, inside of the front door, or may even be provided on the supports 26 themselves. The light sources 18 may be provided within a recess so as to not protrude beyond the walls. The light sources 18 also may be arranged vertically and/or horizontally spaced apart, such as an elongated bulb or several light sources strung together vertically, at each corner of the internal cavity, for example. The number and arrangement of the light sources 18 is not limited to that shown in the illustrated embodiment. For example, if a plurality of objects 15 are arranged in a grid or array on a support 26, then one or more light sources 18 could be associated with each region of the grid or array containing a single object. For example, one or more of a top, bottom, left, right, front, and/or back light source 18 could be associated with each region having a single object 15.

Referring particularly to FIG. 6, a schematic illustration of the decontamination unit 10 is shown, including the internal cavity 14, at least one support 26 within the internal cavity 14 for holding the object(s) 15 to be decontaminated, the heater 16 in thermal communication with the internal cavity 14, and the light source 18 that provides UV light (e.g., UVC) to the internal cavity 14 to irradiate or “dose” the object 15. As discussed above, a controller 20 is configured to control the temperature and preferably also the UV irradiation/exposure within the internal cavity 14 according to a decontamination cycle for decontaminating the object 15.

In exemplary embodiments, the controller 20 is operatively coupled to the heater 16 and the light source 18. As discussed above, for example, the heater 16 may include an electric heating element 30 and a blower 32, both of which may be controlled independently by the controller 20 to achieve the desired temperature in the cavity 14. The light source 18 also may be controlled by the controller 20 independently of the heater 16 to achieve the desired amount or level of irradiation in the cavity for providing exposure or “dosage” to the object(s) 15 to be decontaminated. The controller 20 may control all of the light sources 18 together, or may control one or more of the light sources 18 independently of each other, such as in sets associated with each support 26. Although control of light sources 18 for controlling UV irradiation/exposure preferably is provided by the controller 20, in some embodiments the UV portion of the decontamination cycle may be provided by a user selectively turning the UV lights on and off for the UV cycle time.

The controller 20 may include any suitable apparatus, device(s), or machine(s) for processing data, including a primary control circuit that is configured to carry out various control operations relating to control of the decontamination unit 10. The controller may include by way of example, a programmable processor, a computer, or multiple processors or computers. For example, the primary control circuit may include an electronic processor, such as a CPU, microcontroller, or microprocessor. The operative connection(s) of the controller to the heater 16 and light source 18 includes those in which signals, physical communications, or logical communications may be sent or received. Typically, an operative connection includes a physical interface, an electrical interface, or a data interface, but it is to be noted that an operative connection may include differing combinations of these or other types of connections sufficient to allow operative control.

The controller 20 also may include, in addition to hardware, code that creates an execution environment for the computer program in question. For example, among their functions, to implement the features described herein, the control circuit and/or electronic processor may comprise an electronic controller that may execute program code embodied as the decontamination unit control application. It will be apparent to a person having ordinary skill in the art of computer programming, and specifically in application programming for electronic and communication devices, how to program the device to operate and carry out logical functions and instructions associated with the control application. Accordingly, details as to specific programming code have been left out for the sake of brevity. The decontamination unit control application may be stored in a non-transitory computer readable medium, such as random access memory (RAM), a read-only memory (ROM), an erasable programmable read-only memory (EPROM or Flash memory), or any other suitable medium. It is understood that although instructions for performing the methods described herein may be executed by the processor components of the controller, such controller functionality could also be carried out via dedicated hardware, firmware, software, or combinations thereof.

To provide feedback control to the controller 20, one or more sensors for determining the temperature, and optionally UV exposure, within the internal cavity also are provided and operatively coupled to the controller 20. Any suitable type or types of sensor(s) may be provided for communicating the measured temperature and optionally UV level to the controller for providing control of these parameters. For example, in the illustrated embodiment, at least one temperature sensor 50 is provided in thermal communication with the internal cavity 14, such as a thermostat or thermocouple having a thermal sensing portion within the internal cavity 14. A separate UV sensor 52 (e.g., UVC detector or dosimeter) also may be provided in suitable communication with the internal cavity 14 for measuring and communicating the UV level within the cavity for UV control by the controller 20. Alternatively, no such UV sensor 52 may be provided, and instead exposure may be determined based upon the power of the light source(s) 18, the time of the exposure, or the like. In this scenario, the controller 20 may control the light sources 18 simply by turning them on for a period of time.

The controller 20 includes a user interface 54 that enables the user to start the decontamination cycle. Any suitable user interface may be provided, including one or more physical buttons, switches, knobs, sliders and the like; or an electrical touchscreen display with one or more of the foregoing. Suitable indicator(s) and/or displays, including lights, LCDs, LEDs, etc. also may be provided for displaying information, such as warning indicators, internal cavity temperature, internal cavity UVC level, timer, and the like. The user interface 54 also may include an on/off switch to power on or power off the unit. In exemplary embodiments, the controller 20 may lock-out user interaction and interruption during a decontamination cycle.

The user interface 54 also may include a display for a menu of options to control the decontamination unit in a particular manner, such as selecting a desired decontamination cycle. For example, the controller 20 (or a non-transitory computer readable medium, such as a hard drive) may store a plurality of preset decontamination cycles depending on the objects to be decontaminated and/or the type of contaminant to be removed. Bulky items or a large quantity of items with a higher thermal load, for example, may require a longer cycle due to the thermal mass involved in heating the internal cavity to the setpoint decontamination temperature. Certain viruses bacteria, spores, or other contaminants may utilize different parameters in respective decontamination cycles for decontamination thereof based on their characteristics.

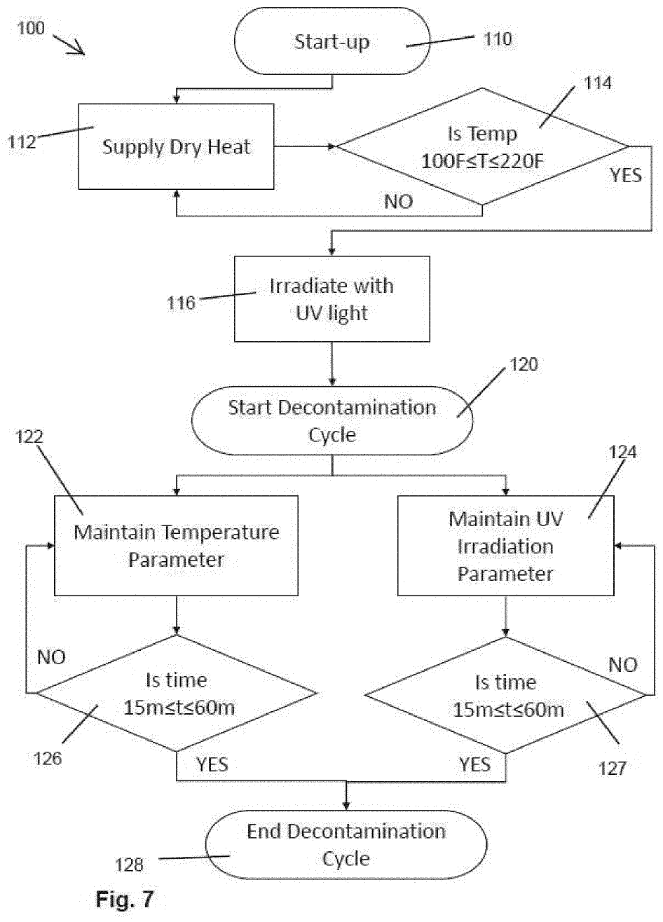

FIG. 7 illustrates a flow chart of an exemplary process 100 including a decontamination cycle that uses the decontamination unit 10 for decontaminating contaminated objects. As noted above, the process may be performed at atmospheric pressure, with reasonably low temperatures, relatively short cycle times, and without the use of chemicals. In exemplary embodiments, the decontamination cycle is a preset cycle that runs a predetermined temperature profile, or setpoint value(s), a predetermined UV output profile, or setpoint value(s), and a predetermined period of time, as described in further detail below. As noted above, once a preset decontamination cycle is started, the controller 20 may lock-out the controls for preventing changing the temperature, UV or time parameters.

As shown in FIG. 7, the process 100 begins at step 110 with a start-up of the decontamination unit 10, which may include powering the unit. At step 112, heat is supplied by the heater 16 to heat the internal cavity 14 to a setpoint temperature for the decontamination cycle. The heat is preferably dry heat. Generally, the dry heat will have a relative humidity level of less than 25% RH, such as between 0% RH to 20% RH, for example. During this time, the temperature may ramp up at a predetermined ramp rate, or as fast as possible depending on the thermal load in the internal cavity 14 and power of the heater 16.

As shown at step 114, in exemplary embodiments the setpoint temperature inside of the internal cavity 14 for the decontamination cycle is in a range from about 100° F. (38° C.) to about 220° F. (140° C.). In some preferred embodiments, the temperature inside of the internal cavity 14 is in a range from about 125° F. (52° C.) to about 212° F. (100° C.), and more particularly about 140° F. to 180° F. As such, the setpoint temperature for the decontamination cycle may be any value, range or subrange between or within the foregoing ranges, such as a temperature of about any of 100° F., 110° F., 120° F., 130° F., 140° F., 150° F., 160° F., 170° F., 180° F., 190° F., 200° F., 210° F., or 220° F. (including any values or subranges between the stated values). The lower temperatures may be particularly suitable for thermally sensitive objects, such as certain electronics, for example, that have a low thermal degradation temperature. The lower temperature setpoints also are faster to achieve. Increased temperatures, however, may increase decontamination rate.

Step 116 shows supplying UV light (e.g., UVC) to the internal cavity with the light source 18. The supply of UV light to the internal cavity 14 at step 116 may occur substantially simultaneously with the supply of heat at step 112; or may be supplied consecutively after the temperature setpoint has been reached. During this time, the UV level may ramp up at a predetermined ramp rate, or as fast as possible, such as during warm-up of the UV bulbs. Although the process 100 shows the UV and heat being used simultaneously, it is understood that the decontamination unit 10 also is capable of independent operation of the heat and UV functions as well. For example, certain preset decontamination cycles may use only heat via heater 16, some decontamination cycles may use only UV via light source(s) 18, some decontamination cycles may use one then switch to the other, or some may use both simultaneously for all or only some of the decontamination cycle.

The light source(s) 18 have a suitable power or output emission (light intensity) to emit a desired level of the light (e.g., UVC light) to decontaminate the object 15 during a time of the decontamination cycle. Generally, destruction or inactivation of the contaminant (e.g., biological contaminant) is based on the dosage of UV light received, which is in terms of energy per area (e.g., mJ/cm²). The dosage to decontaminate the object may be based upon the type of contaminant, the size of the object, the level of contamination, the type of light (e.g., UVA, UVB, UVC, etc.), the intensity of the light, or the like. An exemplary dosage to decontaminate a virus or bacteria, for example, may be in the range from 100 mJ/cm² to 1,000 mJ/cm². A light intensity of the UV light may be in the range of 40 micro-W/cm² to 2,000 micro-W/cm², for example. Of course, with the application of both heat and UV light, the intensity of the light used to achieve decontamination may be less than UV light alone. Likewise, with both UV and heat applied, the temperature during decontamination may be less than using dry heat alone.

As shown at step 120, once the setpoint temperature and UV light values have been achieved inside the internal cavity 14, the decontamination cycle is started. As such, the decontamination cycle and time thereof does not include the ramp up and ramp down times in the exemplary process, but could. The objects to be contaminated may be placed in the internal cavity 14 during the ramp up, or may be placed in the cavity after the setpoints have been reached and before the start of the decontamination cycle.

As shown at steps 122 and 124, both the temperature and the UV light parameters (values) are maintained during the decontamination cycle, which may include some degree of variation depending on the control of these parameters. Generally, the controller 20 uses information from the sensor(s) (e.g., temperature sensor 50 and optionally UV sensor 52) to maintain the temperature and UV level setpoints within the cavity 14 during the decontamination cycle. The controller 20 may use any suitable logic, such as PID-loop logic, to maintain the setpoint value inside of the internal cavity 14 within a certain error band, for example within 10% of the setpoint value.

The decontamination cycle may be configured to run for a predetermined period of time according to the preset conditions, as shown at steps 126 and 127. As noted, the application of heat and UV light may occur simultaneously at this stage, or may be offset, or consecutive to one another. In exemplary embodiments, the decontamination cycle time for each stage of heat and UV light is in a range from 10 minutes to 60 minutes, such as about any of 10, 15, 20, 25, 30, 35, 40, 45, 50, 55 or 60 minutes (including all values, ranges and subranges between the stated values). The total decontamination cycle time including both heat and UV light may be in a range from 10 minutes to 60 minutes, such as about any of 10, 15, 20, 25, 30, 35, 40, 45, 50, 55 or 60 minutes (including all values, ranges and subranges between the stated values). By way of example and not limitation, at a total decontamination cycle time of 25 minutes, the temperature could satisfy its value(s) in its above-noted range (e.g., 140° F.) for the full 25 minutes, whereas the UV level might satisfy its value(s) in its range for only 15 minutes of the 25 minute cycle. Generally, shorter periods of time are particularly advantageous to increase throughput of decontaminating contaminated objects. After the time has been satisfied, the decontamination cycle ends at step 128 and the object(s) are decontaminated.

As noted above, the heat provided by the heater 16 preferably is in the form of dry heat to prevent moisture or condensation on the light sources 18, and also to enable decontamination of electronics, such as laptops, cellphones, or the like.

Also noted above, in exemplary embodiments the decontamination cycle is performed without the use of chemicals, such that the exemplary unit is a chemical-free decontamination unit. A chemical-free decontamination unit and method provides advantages in terms of not having to handle or dispense of the chemicals, nor have separate devices to supply chemicals to the internal cavity 14. The use of chemicals, of course, may aid in the decontamination or sterilization of the object(s), and thus could be employed in some embodiments.

Further noted above, in exemplary embodiments the decontamination is performed at atmospheric pressure, which provides advantages in terms of reducing complexity of the unit 10, cycle time, etc. The use of positive pressure, of course, may provide advantages in terms of decontamination, and thus may be applied in some embodiments.

As noted above, different viruses, bacteria, spores, or other contaminants may utilize different decontamination parameters for decontamination thereof based upon the characteristics of the contaminant. Decontamination, or disinfection, is quantified by inactivation rates or Log Reduction Value (or LRV). Log reduction is a simple mathematical term used to express the relative number of live microbes eliminated by disinfection. A 3 log reduction equates to 99.9%, a 4 log reduction equates to 99.99%, and a 6 log reduction equates to 99.9999%. Generally it is desirable to provide a greater than 6 log reduction (99.9999%) from virus, bacteria and spores. This could include, for example, MS2 Bacteriophage (non-lipid surrogate for Sars-Cov-2), Mycobacteria, or difficult to kill spores (C. difficile spores).

It is understood by the inventors that a decontamination cycle with the parameters and ranges identified in the exemplary flow chart of FIG. 7 are effective in decontaminating contaminated objects. One exemplary method understood to eradicate MRSA or E. coli is a total decontamination cycle time of 25 minutes, with a heat cycle that runs for a preset time of 25 minutes at 140° F. and a UVC cycle that runs for a preset time of 15 minutes within the 25 minute total time (e.g., the end portion of the total time). As noted above, the process may be performed at atmospheric pressure, with reasonably low temperatures using dry heat, relatively short cycle times, and without the use of chemicals. Once a preset decontamination cycle is started, the controller 20 may lock-out the controls for preventing changing the temperature, UV, or time parameters. The controller also may use a magnetic lock to lock the door and prevent access to the internal cavity (and exposure of the UV light to a user); or may be configured to terminate the cycle/prevent startup if the door is opened.

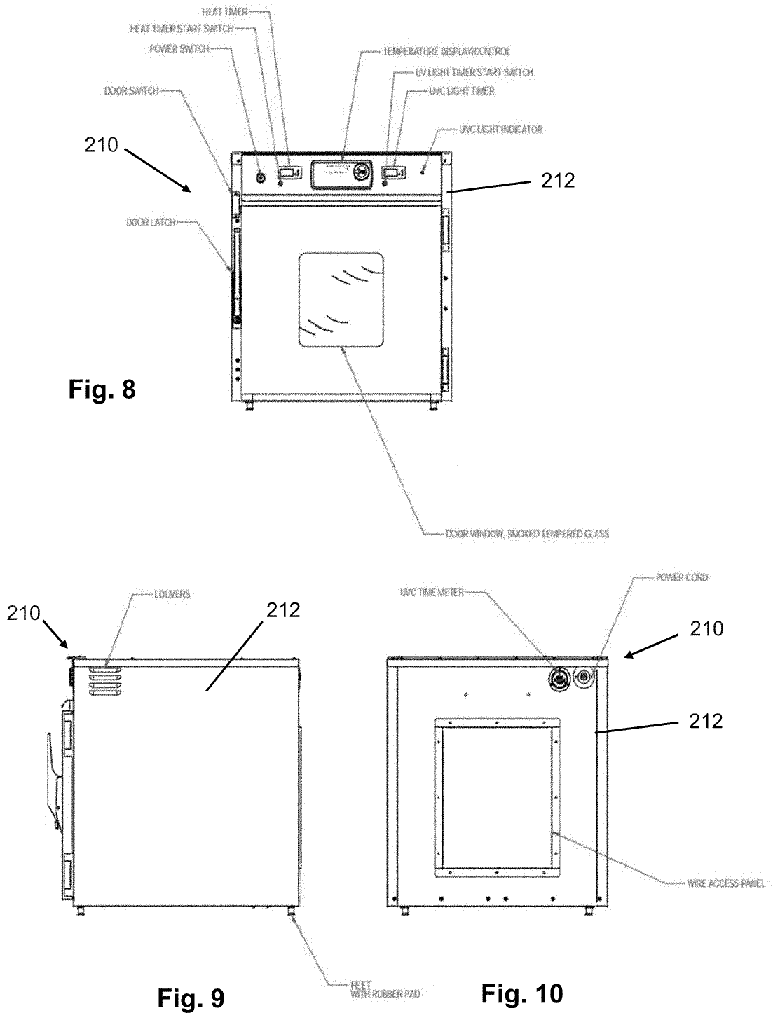

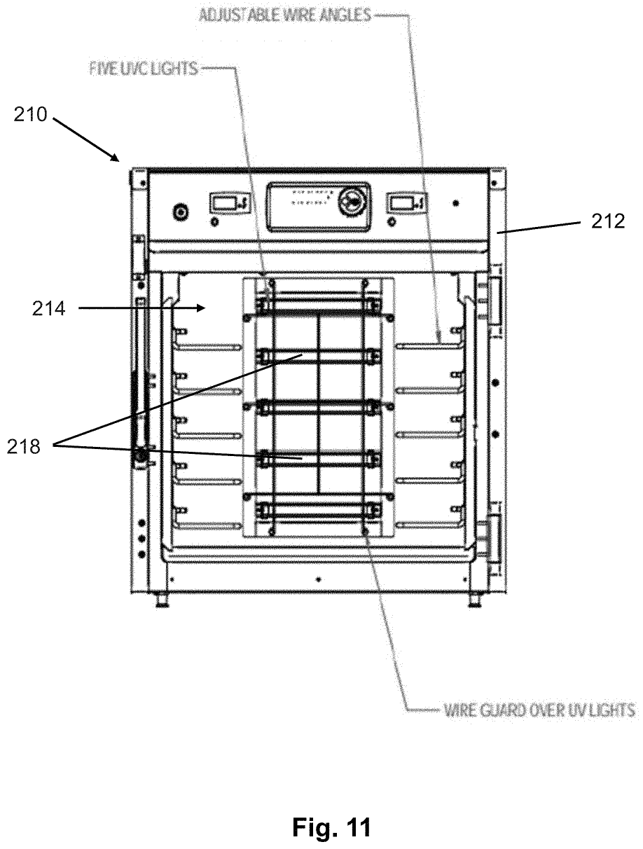

Turning to FIGS. 8-11, another exemplary embodiment of a decontamination unit 210 is shown. The decontamination unit 210 is substantially similar to the above-referenced decontamination unit 10, and consequently the same reference numerals, but in the 200-series, are used to denote structures corresponding to similar structures. In addition, the foregoing description of the decontamination unit 10 is equally applicable to the decontamination unit 210, except as understood by the description below. It is also understood that aspects of the decontamination units 10, 210 may be substituted for one another or used in conjunction with one another where applicable.

As shown in the exemplary embodiment, the decontamination unit 210 is essentially a smaller (e.g., tabletop) version of the decontamination unit 10. As such, the decontamination unit 210 includes a housing 212 having an internal cavity 214 for containing a contaminated object that is to be decontaminated. The decontamination unit 210 also includes a heater (hidden from view) that is in thermal communication with the internal cavity 214 and is configured to heat the internal cavity 214 and object, preferably with dry heat. The decontamination unit 210 also includes at least one light source 218, and preferably multiple light sources 218, configured to supply ultraviolet light (e.g., UVC light) to the internal cavity to irradiate the object. A controller (not shown) is provided that is configured to control a temperature and preferably also the UV irradiation/exposure within the internal cavity 214 according to a decontamination cycle for decontaminating the object. The decontamination cycle may be performed at atmospheric pressure. In exemplary embodiments, the decontamination cycle may be performed free from the use of chemicals.

Exemplary decontamination units and methods have been described herein. The exemplary decontamination units include a housing having an internal cavity for containing an object that is to be decontaminated, a heater that is configured to heat the internal cavity and object, and at least one light source configured to supply ultraviolet light, such as UVC, to the internal cavity to irradiate the object. An exemplary decontamination cycle may perform both the heat cycle and UV cycle simultaneously during at least a part of the cycle to enhance the effectiveness in decontaminating the object.

As is apparent from the foregoing description, the exemplary dry heat and/or UV light decontamination unit and method includes many advantages, including one or more advantages of: (i) a relatively low-temperature dry heat decontamination cycle combined with an associated level of UV-light, such as UVC, for decontaminating an object; (ii) decontamination at atmospheric pressure; (iii) decontamination without the use of chemicals, which may be particularly advantageous for electronic devices, for example; (iv) an arrangement of the UV light sources relative to the object-supports that minimizes shadowing and enhances decontamination; (v) a relatively short cycle time for increasing decontamination throughput of contaminated objects, which may be particularly advantageous in the event of a pandemic, for example; and/or (vi) a relatively inexpensive unit that is affordable and deployable at various points of use, such as at schools, police stations, fire stations, department of defense, other government agencies, elderly care facilities, hospitals, or the like.

Specifically, research by the inventors indicates that significantly no appropriate localized solution has been found to exist for decontamination of objects, including everyday electronics such as laptops, computer accessories, cell phones, tablets, etc., beyond merely wiping down and spraying occasionally.

The exemplary decontamination unit and method described herein may thus provide improvements including one or more of: (i) decontaminating electronics such as laptops, computer accessories, cell phones, radio, tablets, etc.; (ii) flexibility to choose from heat and/or UV (e.g., UVC) to decontaminate items that are sensitive to high heat, and/or providing the heat as dry heat for decontaminant items that are sensitive to moisture including but not limited to linen, electronics, books, etc.; (iii) using heat up to 140° F., or beyond where applicable, to decontaminate objects; (iv) using UV light, such as UVC, (e.g., at 254 nm) to decontaminate objects; (v) providing the UV light and heat independently or together; (vi) providing a preset decontamination cycle for decontaminating an object to ensure ease of use and repeatability; (vii) providing a relatively low temperature and short decontamination time with the addition of UV to provide a cycle such as 140° F. for 25 minutes of heat cycle and a 15 min cycle for UVC; (viii) allowing the heat, UV and time parameters to be changed by the user by entering the setup mode if desires; (xi) providing a breaker, such as a magnetic circuit breaker, to automatically turn off the UV lights when the door is opened; (x) providing a UV hour meter to continuously record the number of hours the light has been used to enable periodic replacement ensuring proper light output and maintain high decontamination efficiency; (xi) providing additional lock to prevent accidental door opening during the decontamination cycle; (xii) providing site glass that blocks UV light; (xiii) providing indicators that cycle is on, auto cut off when door is opened, bulb guard protects the lights from being damaged; (xiv) preferably uses no chemicals or ozone, hence no residual toxicity or odor ensuring long term health safety by items placed; (xv) or the like.

According to an aspect a decontamination unit includes: a housing having an internal cavity and an opening for accessing the internal cavity; a door coupled to the housing for opening or closing the opening for permitting or restricting access to the internal cavity; a heater in thermal communication with the internal cavity and configured to heat the internal cavity; and a light source configured to supply ultraviolet light to the internal cavity; wherein the decontamination unit is configured to enable each of (i) heating the internal cavity with the heat source, and (ii) irradiating the internal cavity with the light source.

Embodiments may include one or more of the following additional features, separately or in any combination.

In some embodiments, a decontamination cycle of the decontamination unit is configured to: (i) heat the internal cavity with the heater absent irradiating the internal cavity with the light source; (ii) irradiate the internal cavity with the light source absent heating the internal cavity with the heat source; (iii) heat the internal cavity with the heater before or after irradiating the internal cavity with the light source; or (iv) both (i) heat the internal cavity with the heat source and (ii) irradiate the internal cavity with the light source simultaneously during at least a portion of the decontamination cycle of the unit.

In some embodiments, the UV light is UVC light emitted by a UVC light source.

In some embodiments, the wavelength range of the UV light is from 200 nm to 300 nm.

In some embodiments, the wavelength range of the UV light is about 254 nm.

In some embodiments, the heater is configured to supply dry heat and wherein the decontamination unit runs a decontamination cycle with the dry heat.

In some embodiments, the dry heat has a relative humidity of less than 25%, such as in a range from 0% RH to 20%.

In some embodiments, the decontamination unit, further comprising: a plurality of supports configured to support a plurality of objects for decontamination; wherein the light source is at least one of a plurality of light sources within the internal cavity, each of the plurality of light sources being configured to irradiate a respective portion of the cavity; and wherein each support of the plurality of supports has associated therewith at least one light source of the plurality of light sources.

In some embodiments, more than one support share at least one of the plurality of light sources, in particular, wherein at least one light source is arranged vertically within the chamber at a corner and extends upwardly to irradiate a plurality of the supports, such as from a top to a bottom of the chamber.

In some embodiments, two or more of the corners of the chamber, such as all four corners, include a vertically oriented light source configured to irradiate a plurality of the supports, for example all four light sources extending from a top to bottom of the chamber.

In some embodiments, the plurality of supports include a plurality of vertically spaced apart horizontal supports, and wherein the associate at least one light source of each support is at an elevation above its corresponding horizontal support.

In some embodiments, a second light of the plurality of lights is provided at an elevation below each horizontal support, the horizontal support having openings or being transparent to the wavelength of the UV light to irradiate an underside of the object that is supported by the horizontal support.

In some embodiments, the decontamination unit further comprising: a controller operatively coupled to the heat source the controller being configured to control a temperature within the internal cavity according to a preset decontamination cycle.

In some embodiments, the controller is operatively coupled to the light source or plurality of light sources, the controller being configured to control irradiation of the light source(s) according to the preset decontamination cycle.

In some embodiments, a decontamination cycle runs for a total cycle time and includes a heat cycle portion and/or a UV cycle portion, and
wherein the decontamination cycle performs the heat cycle alone;
wherein the decontamination cycle performs the UV cycle alone;
wherein the heat cycle is started before the UV cycle,
wherein the heat cycle is started after the UV cycle,
wherein the heat cycle and the UV cycle are started at the same time,
wherein the heat cycle is ended before the UV cycle,
wherein the heat cycle is ended after the UV cycle, or
wherein the heat cycle and the UV cycle are ended at the same time.

In some embodiments, the decontamination unit is configured to run a decontamination cycle having at least the following parameters: a temperature in a range from 100° F. to 220° F. for a time of 10-60 minutes; and/or a UV exposure for a time of 10-60 minutes.

In some embodiments, the decontamination unit is configured to run a decontamination cycle for a total cycle time, the decontamination cycle having at least the following parameters: a temperature in a range from 100° F. to 220° F. for a time of 25 minutes within the total cycle time; and/or a UV exposure with UVC light at 254 nm for a time of 15 minutes within the total cycle time.

In some embodiments, a decontamination cycle is performed without the use of a chemical or chemicals.

In some embodiments, a decontamination cycle is performed at ambient pressure.

In some embodiments, a controller locks adjustment to a preset decontamination cycle when the decontamination cycle is running.

According to another aspect, a method of decontaminating an object includes: placing the object within an internal cavity of a decontamination unit; supplying heat to the internal cavity and increasing the temperature of the internal cavity to a temperature value in a range from 100° F. to 220° F.; supplying UV light to the internal cavity; and performing decontamination with the supplying heat and the supplying UV light for a period of time in a range from 15 minutes to 60 minutes.

Embodiments of the subject matter described in this disclosure can be implemented in combination with digital electronic circuitry, controllers, processors, computer software, firmware, and/or hardware. For example, embodiments may be implemented in a decontamination unit that uses one or more modules of computer program with instructions encoded on a non-transitory computer-readable medium for execution by, or to control the operation of, data processing apparatus. The operations may include physical manipulations of physical quantities. Usually, though not necessarily, the physical quantities take the form of electrical or magnetic signals capable of being stored, transferred, combined, compared, and otherwise manipulated in a logic and the like.

The controller may include, in addition to hardware, code that creates an execution environment for the computer program in question. The computer program (also referred to as software or code), may be deployed in any form, including as a stand-alone program or as a module, component, subroutine, or other unit suitable for use in a computing environment. The computer program may be deployed to be executed on one computer or on multiple computers that are located at one site or distributed across multiple sites and interconnected by a communication network. The processor may include all apparatus, devices, and machines suitable for the execution of a computer program, which may include, by way of example, both general and special purpose microprocessors, and any one or more processors of any kind of digital computer. Generally, the processor will receive instructions and data from a read-only memory or a random-access memory or both. The computer may include, or be operatively coupled to receive data from or transfer data to, or both, one or more mass storage devices for storing data. Devices suitable for storing computer program instructions and data include all forms of non-volatile memory, media and memory devices, including by way of example semiconductor memory devices. The processor and the memory can be supplemented by, or incorporated in, special purpose logic circuitry.

In the flow diagram(s), blocks denote “processing blocks” that may be implemented with logic. The processing blocks may represent a method step or an apparatus element for performing the method step. A flow diagram does not depict syntax for any particular programming language, methodology, or style (e.g., procedural, object-oriented). Rather, a flow diagram illustrates functional information one skilled in the art may employ to develop logic to perform the illustrated processing. It will be appreciated that in some examples, program elements like temporary variables, routine loops, and so on, are not shown. It will be further appreciated that electronic and software applications may involve dynamic and flexible processes so that the illustrated blocks can be performed in other sequences that are different from those shown or that blocks may be combined or separated into multiple components. It will be appreciated that the processes may be implemented using various programming approaches like machine language, procedural, object oriented or artificial intelligence techniques. In one example, methodologies are implemented as processor executable instructions or operations provided on a computer-readable medium. Thus, in one example, a computer-readable medium may store processor executable instructions operable to perform a method.

As used herein, an “operative connection,” or a connection by which entities are “operatively connected,” is one in which the entities are connected in such a way that the entities may perform as intended. An operative connection may be a direct connection or an indirect connection in which an intermediate entity or entities cooperate or otherwise are part of the connection or are in between the operatively connected entities. An “operative connection,” also may be one in which signals, physical communications, or logical communications may be sent or received. Typically, an operative connection includes a physical interface, an electrical interface, or a data interface, but it is to be noted that an operative connection may include differing combinations of these or other types of connections sufficient to allow operative control. For example, two entities can be operatively connected by being able to communicate signals to each other directly or through one or more intermediate entities like a processor, operating system, a logic, software, or other entity. Logical or physical communication channels can be used to create an operable connection.

It is to be understood that terms such as “top,” “bottom,” “upper,” “lower,” “left,” “right,” “front,” “rear,” “forward,” “rearward,” and the like as used herein may refer to an arbitrary frame of reference, rather than to the ordinary gravitational frame of reference.

It is to be understood that all ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The term “about” as used herein refers to any value which lies within the range defined by a variation of up to ±10% of the stated value, for example, ±10%, ±9%, ±8%, ±7%, ±6%, ±5%, ±4%, ±3%, ±2%, ±1%, ±0.01%, or ±0.0% of the stated value, as well as values intervening such stated values.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A decontamination unit comprising:
a housing having an internal cavity and an opening for accessing the internal cavity;
a door coupled to the housing for opening or closing the opening for permitting or restricting access to the internal cavity;
a heater in thermal communication with the internal cavity and configured to heat the internal cavity; and
a light source configured to supply ultraviolet light to the internal cavity;
wherein the decontamination unit is configured to enable each of (i) heating the internal cavity with the heat source, and (ii) irradiating the internal cavity with the light source.

2. The decontamination unit according to claim 1, wherein a decontamination cycle of the decontamination unit is configured to:
(i) heat the internal cavity with the heater absent irradiating the internal cavity with the light source;
(ii) irradiate the internal cavity with the light source absent heating the internal cavity with the heat source;
(iii) heat the internal cavity with the heater before or after irradiating the internal cavity with the light source; or
(iv) both (i) heat the internal cavity with the heat source and (ii) irradiate the internal cavity with the light source simultaneously during at least a portion of the decontamination cycle of the unit.

3. The decontamination unit according to claim 1, wherein the UV light is UVC light emitted by a UVC light source.

4. The decontamination unit according to claim 1, wherein the wavelength range of the UV light is from 200 nm to 300 nm.

5. The decontamination unit according to claim 1, wherein the wavelength range of the UV light is about 254 nm.

6. The decontamination unit according to claim 1, wherein the heater is configured to supply dry heat and wherein the decontamination unit runs a decontamination cycle with the dry heat.

7. The decontamination unit according to claim 6, wherein the dry heat has a relative humidity of less than 25%, such as in a range from 0% RH to 20%.

8. The decontamination unit according to claim 1, further comprising:
a plurality of supports configured to support a plurality of objects for decontamination;
wherein the light source is at least one of a plurality of light sources within the internal cavity, each of the plurality of light sources being configured to irradiate a respective portion of the cavity; and
(i) wherein each support of the plurality of supports has associated therewith at least one light source of the plurality of light sources; and/or
(ii) wherein more than one support share at least one of the plurality of light sources, in particular, wherein at least one light source is arranged vertically within the chamber at a corner and extends upwardly to irradiate a plurality of the supports, such as from a top to a bottom of the chamber, more particularly wherein two or more of the corners of the chamber, such as all four corners, include a vertically oriented light source configured to irradiate a plurality of the supports, for example all four light sources extending from a top to bottom of the chamber.

9. The decontamination unit according to claim 8, wherein the plurality of supports include a plurality of vertically spaced apart horizontal supports, and wherein the associate at least one light source of each support is at an elevation above its corresponding horizontal support.

10. The decontamination unit according to claim 9, wherein a second light of the plurality of lights is provided at an elevation below each horizontal support, the horizontal support having openings or being transparent to the wavelength of the UV light to irradiate an underside of the object that is supported by the horizontal support.

11. The decontamination unit according to claim 1, further comprising:
a controller operatively coupled to the heat source the controller being configured to control a temperature within the internal cavity according to a preset decontamination cycle.

12. The decontamination unit according to claim 11, wherein the controller is operatively coupled to the light source or plurality of light sources, the controller being configured to control irradiation of the light source(s) according to the preset decontamination cycle.

13. The decontamination unit according to claim 1, wherein a decontamination cycle runs for a total cycle time and includes a heat cycle portion and/or a UV cycle portion, and
wherein the decontamination cycle performs the heat cycle alone;
wherein the decontamination cycle performs the UV cycle alone;
wherein the heat cycle is started before the UV cycle,
wherein the heat cycle is started after the UV cycle,
wherein the heat cycle and the UV cycle are started at the same time,
wherein the heat cycle is ended before the UV cycle,
wherein the heat cycle is ended after the UV cycle, or
wherein the heat cycle and the UV cycle are ended at the same time.

14. The decontamination unit according to claim 1, wherein the decontamination unit is configured to run a decontamination cycle having at least the following parameters:
a temperature in a range from 100° F. to 220° F. for a time of 10-60 minutes; and/or
a UV exposure for a time of 10-60 minutes.

15. The decontamination unit according to claim 1, wherein the decontamination unit is configured to run a decontamination cycle for a total cycle time, the decontamination cycle having at least the following parameters:
a temperature in a range from 100° F. to 220° F. for a time of 25 minutes within the total cycle time; and/or
a UV exposure with UVC light at 254 nm for a time of 15 minutes within the total cycle time.

16. The decontamination unit according to claim 1, wherein a decontamination cycle is performed without the use of a chemical or chemicals.

17. The decontamination unit according to claim 1, wherein a decontamination cycle is performed at ambient pressure.

18. The decontamination unit according to claim 1, wherein a controller locks adjustment to a preset decontamination cycle when the decontamination cycle is running.

19. A method of decontaminating an object, comprising:
placing the object within an internal cavity of a decontamination unit;
supplying heat to the internal cavity and increasing the temperature of the internal cavity to a temperature value in a range from 100° F. to 220° F.;
supplying UV light to the internal cavity; and
performing decontamination with the supplying heat and the supplying UV light for a period of time in a range from 15 minutes to 60 minutes.

20. (canceled)
